(19) United States
(12) Patent Application Publication
(10) Pub. No.: US 2008/0081791 A1
(43) Pub. Date: Apr. 3, 2008

(54) Methods of using combinations of siRNAs for treating a disease or a disorder, and for enhancing siRNA efficacy in RNAi

(76) Inventors: Weida Huang, Shanghai (CN); Jie Hong, Yunnan Province (CN); Zhikang Qian, Shanghai (CN)

Correspondence Address: FISH & RICHARDSON PC, P.O. BOX 1022, MINNEAPOLIS, MN, 55440-1022, US

(21) Appl. No.: 11/481,879

(22) Filed: Jul. 6, 2006

(51) Int. Cl. A61K 31/7052; A61P 35/00; A61P 43/00; C07H 21/02; C12N 15/00

(52) U.S. Cl. 514/44; 435/455; 536/23.1

(57) ABSTRACT

The present invention provides methods for treating diseases or disorders, and methods for enhancing siRNA efficacy in RNAi, including administering to a subject or a biological system one or more siRNAs capable of down regulating the expression of one or more target genes and one or more siRNAs capable of down regulating the expression of one or more negative regulators of RNAi. The present invention also provides compositions including one or more siRNAs, or precursors thereof, capable of down regulating the expression of one or more target genes and comprising one or more siRNAs, or precursors thereof, capable of down regulating the expression of one or more negative regulators of RNAi.

### FIELD OF THE INVENTION

The present invention relates to the fields of therapeutics and molecular biology concerning RNAi and siRNA. Specifically, the present invention provides methods for treating a disease or a disorder and methods for enhancing siRNA efficacy, and provides compositions useful in treating a disease or a disorder and in enhancing siRNA efficacy. Some embodiments of the present invention provide methods for treating diseases, such as melanoma and hepatitis B.

### BACKGROUND OF THE INVENTION

The following is a brief description of RNA interference (RNAi) and small interfering RNA (siRNA), and the use thereof in treating diseases. The discussion is provided only for understanding the invention that follows. This summary is not an admission that any of the work described below is prior art to the claimed invention.

RNAi (RNA interference) is a widely conserved phenomenon of post transcriptional gene silencing (PTGS) among nearly all eukaryotes, in which double-stranded RNA (dsRNA) induces the sequence-dependent degradation of cognate mRNA in the cytoplasm, which results in down regulation of the expression of corresponding gene (see Fire et al., Nature (London). 391:806-811 (1998); Bosher and Labouesse, Nat. Cell Biol. 2:E31-E36 (2000); Elbashir et al., Nature (London). 411:494-498 (2001); and Dykxhoorn et al., Nat. Rev. Mol. Cell. Biol. 4:457-467 (2003)). The RNAi phenomenon was initially reported in transgenic plants in 1990. In the following years, RNAi was also observed in almost all eukaryotes including Caenorhabditis elegans, Drosophila, zebrafish and mouse.

The fundamental principles of the mechanism of RNAi have been established in Drosophila. Once introduced into a cell or transcribed from a transgene, dsRNA is first cleaved by Dicer, a member of the RNase III family, into small interfering RNAs (siRNAs) approx. 21-23 nucleotides in length, containing a two-nucleotide overhang at the 3′ end of each strand (see Bernstein et al., Nature (London). 409:363-366 (2001)). Then, siRNA is enzymatically separated into single-stranded RNA molecules and is guided into RISC(RNA-induced Silencing Complex) to form a new complex. Next, the single-stranded RNA in the RISC guides the complex to find and degrade its cognate mRNA (see Hammond et al., Nature (London). 404:293-296 (2000)). The expression of corresponding genes is thus down regulated or suppressed. In RNAi, siRNA plays a key role.

RNAi is thought to have an important role in eliminating invasive viruses in plants and in regulating gene expression during the development of Caenorhabditis elegans and mice. In addition to its physiological role in various eukaryotes, RNAi has proven to be a powerful tool to knock down specific genes in vitro and in vivo, and siRNA is believed to be a powerful tool in treating diseases related to abnormal expression of certain genes, wherein the genes can be either human genes or viral genes.

RNAi regulates the expression of downstream target genes, but the interference itself is also thought to be under regulation. For example, ADARs (adenosine deaminases acting on RNA) and a highly conserved exonuclease-activity-containing protein ERI-1 (enhanced RNAi), whose homologs in human and mouse are named THEX-1 (also called MERI-1 in mouse), discovered in C. elegans have been suggested to be involved in RNAi regulation (see Yang et al., J. Biol. Chem. 280:3946-3953 (2005); Knight and Bass, Mol. Cell. 10:809-817 (2002); Tonkin and Bass, Science. 302:1725 (2003); and Kennedy et al., Nature (London). 427:645-649 (2004)). However, the mechanisms of RNAi regulation have not been elucidated. Accordingly, understanding the mechanisms of RNAi regulation would be essential to develop efficient therapeutic, diagnostic and research uses of RNAi. Thus there is a need in this field to understand and make use of the mechanisms of RNAi regulation. Understanding the mechanisms of RNAi regulation would be useful in methods of using siRNA with enhanced efficacy.

### SUMMARY OF THE INVENTION

The present invention provides, in one embodiment, a method for treating a disease or a disorder. The methods include administering to a subject one or more siRNAs capable of down regulating the expression of one or more target genes and one or more siRNAs capable of down regulating the expression of one or more negative regulators of RNAi.

In other embodiments of the present invention, methods are provided for enhancing siRNA efficacy. The methods include administering to a biological system, e.g., a cell or an animal, one or more siRNAs capable of down regulating the expression of one or more target genes and one or more siRNAs capable of down regulating the expression of one or more negative regulators of RNAi.

In other embodiments of the present invention, compositions are provided that include one or more siRNAs, or precursors thereof, capable of down regulating the expression of one or more target genes, and also including one or more siRNAs, or precursors thereof, capable of down regulating the expression of one or more negative regulators of RNAi.

In yet other embodiments, the present invention provides methods for determining an optimal ratio of siRNAs capable of down regulating the expression of one or more target genes to siRNAs capable of down regulating the expression of one or more negative regulators of RNAi in methods for treating a disease or a disorder and in methods for enhancing siRNA efficacy. The methods can include the following steps, in any order:
a) inducing the expression of genes encoding the negative regulators of RNAi using any siRNA molecules;
b) determining the effective dose of siRNA molecules that is able to induce high expression of negative regulators of RNAi;
c) based on the high expression of negative regulators of RNAi in (b), determining the dose of the siRNA that down regulates expression of the negative regulators of RNAi to base expression level; and
d) based on the down regulation of negative regulators of RNAi in (c), determining the dose of the siRNA that down regulates expression of one or more target genes to the lowest level.

According to methods provided by some embodiments of the present invention, siRNAs targeting thex1 gene, and/or any other gene(s) encoding negative regulators of RNAi, can be used in combination with siRNAs targeting a target gene to significantly improve the therapeutical effects or efficacy of the siRNAs targeting a target gene. Methods provided herein can further reduce the administration dose of the siRNAs targeting a target gene in therapeutic uses, thus the cost of the treatment may be reduced. The methods provided herein are powerful methods for treating cancers, viral diseases, and any disease related to abnormal expression of normal genes.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a map of pET-loop, a plasmid vector expressing dsRNA with stem loop structure.

FIG. 2 shows a map of pET-loop-2C-MYC.

FIG. 3 shows a map of pET-loop-2HBVP.

FIG. 4 shows a map of pET-loop-2MERI-1.

FIG. 5 shows a map of pET-loop-2MADAR1

FIG. 6 shows dsRNA purified with a CF-11 column. Lane 1: E. coli RNA extraction containing pET-loop-2HBVP or pET-loop-2MERI-1. Lane 2: E. coli RNA extraction containing pET-loop. Lane 3: CF-11 column purified sample of E. coli RNA extraction containing pET-loop-2HBVP or pET-loop-2MERI-1. Lane 4: CF-11 column purified sample of E. coli RNA extraction containing pET-loop.

FIGS. 7A and 7B show the preparation and purification of esiRNA (Escherichia-coli-expressed and enzyme-digested siRNAs). 7A: the effect of different quantities of His-RNaseIII on hydrolysis of dsRNA. 0, 0.1 μg, 0.25 μg, 0.5 μg, 1 μg, 2 μg or 4 μg His-RNaseIII is used in lanes 1-7, respectively; 7B: the purification of 21-23 bp esiRNA on Superdex-75 column.

FIG. 8 shows the suppression of HBsAg expression by esiHBVP in CHO-iHBS cells.

FIG. 9 shows the relative HbsAg level in the serum of mice transfected with different quantities of esiHBVP.

FIGS. 10A-D shows an RT-PCR analysis of thex-1 and adar-1 gene expression in mice livers injected with different doses of siRNAs. 10A and 10B: Typical electrophoretic profiles of thex-1 and adar-1 amplification products on agarose gels respectively. 10C and 10D: Statistical analysis of mRNA levels of thex-1 and adar-1 determined by densitometric analysis of respective bands in three independent experiments. Each bar represents an average of measurements from more than six mice. Results are mean±S.E.M.*P<0.05, significantly different from the corresponding controls.

FIG. 11 shows the relative HbsAg level in serum of mice transfected with esiHBVP in combination with esiMERI-1.

FIG. 12 shows a diagram of melanoma growth in mice after transfection of different amount of esiC-MYC with or without esiMERI-1 or esiMADAR-1.

### DETAILED DESCRIPTION OF THE INVENTION

The present invention is based on the surprising discovery that a relatively higher dose of purified siRNAs had a suppressive effect of shorter duration than a lower dose of siRNAs, in both cell culture and animal models.

By hypothesizing that high dose siRNA in cells induces down regulation of RNAi by, for example, up regulation of negative regulators of RNAi, including THEX1 and ADAR1, the inventors made a further surprising discovery that the expression of negative regulators of RNAi was also regulated by RNAi, e.g., the expression level of thex1 gene was reduced by siRNA targeting thex1.

Accordingly, the invention in some embodiments provides methods for treating a disease or a disorder. The methods include administering to a subject one or more siRNAs capable of down regulating the expression of one or more target genes, and one or more siRNAs capable of down regulating the expression of one or more negative regulators of RNAi.

In some embodiments, the present invention also provides methods for enhancing siRNA efficacy. The methods include administering to a biological system one or more siRNAs capable of down regulating the expression of one or more target genes and one or more siRNAs capable of down regulating the expression of one or more negative regulators of RNAi.

As used herein, the term “RNAi” refers to RNA interference, which is a widely conserved phenomenon of post transcriptional gene silencing (PTGS) among nearly all eukaryotes, in which double-stranded RNA (dsRNA) induces the sequence-dependent degradation of cognate mRNA in the cytoplasm, resulting in down regulation (or suppression) (“interference”) of the expression of corresponding genes.

As used herein, the term “siRNA” refers to small interfering RNA, or any ribonucleic acid-based molecule which is not more than 30 nucleotides (nt) in length and induces RNAi in vivo and/or in vitro. Preferably, siRNA comprises between 21 and 27 bases complementary to an RNA molecule and induces RNAi, for example, to down-regulate the expression of a target gene, i.e., the gene generating the complementary RNA. Even more preferably, siRNA comprises between 21 and 23 bases complementary to an RNA molecule.

As used herein, “complementary to” means a nucleic acid is able to form hydrogen bond(s) with another nucleic acid by either traditional Watson-Crick or other non-traditional patterns. In other words, these two nucleic acids bind to each other by forming base pairing between them. As is well recognized in the art, traditional Watson-Crick base pairing patterns refer to binding between adenosine and thymidine or uridine by forming two hydrogen bonds between their bases; and binding between guanosine and cytidine by forming three hydrogen bonds between their bases. Non-traditional base pairing patterns include binding between nucleoside pairs, such as adenosine-inosine binding, cytidine-inosine binding, and the like.

As used herein, the term “target gene” refers to a gene from which an RNA molecule complementary to either strand of the administered siRNA is transcribed, and the expression level of the gene is down regulated by the complementary siRNA.

As used herein, the term “down regulate” means that the expression of a gene, or level of RNAs or equivalent RNAs encoding one or more protein subunits, or activity of one or more protein subunits, in a cell or subject, is reduced below that observed in the absence of the nucleic acid molecules administered to the cell or subject.

As used herein, the term “negative regulator of RNAi” refers to a biological molecule, such as a protein or an RNA molecule, whose action has an inhibitory effect on RNAi.

In some embodiments, the negative regulators of RNAi are selected from a group consisting of exonucleases and adenosine deaminases.

In some embodiments, the exonuclease is THEX1 or a homolog thereof.

As used herein, the term “exonuclease” refers to an enzyme that cleaves nucleotide bases sequentially from the free ends of a nucleic acid. An siRNA molecule can be degraded by the exonuclease and thus loses its function.

As used herein, the term “homolog,” when referring to a protein or polypeptide, means that an amino acid sequence of two or more protein or polypeptide molecules is partially or completely identical.

In some preferred embodiments, the adenosine deaminase is ADAR1 or homolog thereof.

As used herein, the term “enhancing siRNA efficacy” means that the same level of suppressive effects of an siRNA is obtained with less corresponding siRNA molecules, or stronger suppressive effects of an siRNA is obtained with the same amount of corresponding siRNA molecules.

As used here in, the term “administer” or “administration” refers to delivering nucleic acids to a subject or any biological system as required. Alternatively, the nucleic acid molecules (e.g., siRNAs) can be expressed from DNA and/or RNA vectors that are delivered to the subject or the biological system.

Methods for the delivery of nucleic acid molecules are well known in the art. For example, nucleic acid molecules can be administered by a variety of methods including, but not limited to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination can be locally delivered by direct injection or by use of an infusion pump. Other approaches include the use of various transport and carrier systems, for example, through the use of conjugates and biodegradable polymers.

As used herein, the term “subject” refers to a human or a non-human animal to which the nucleic acid molecules of the invention can be administered. Preferably, the subject is a human. Where a subject is a human or a non-human animal, the subject will in many cases be in need of treatment.

As used herein, the term “biological system” refers to an in vivo or an in vitro system that includes gene expression machinery, by which a gene carried by a DNA segment can be expressed. In some embodiments, the biological system is an animal, a plant, a cell line, a cell (e.g., a primary or cultured cell), or an artificial gene expression system.

In some embodiments, the ratio of siRNAs capable of down regulating the expression of target genes to siRNAs capable of down regulating the expression of negative regulators of RNAi is in a range of about 5:1 to about 20:1 (w/w). In some embodiments, the ratio of the siRNAs capable of down regulating the expression of target genes to the siRNAs capable of down regulating the expression of negative regulators of RNAi is about 10:1 (w/w).

In some embodiments, the siRNAs are administered at the same time. However, therapeutic nucleic acid molecules (e.g., siRNA) delivered exogenously may be stable and retain their activity within the body of the subject for a certain period. This period of time varies between hours to days. For example, such a period can be 3 days. Therefore, in some alternative embodiments, the siRNAs capable of down regulating the expression of target genes are administered after the siRNAs capable of down regulating the expression of negative regulators of RNAi have been administered, and while they still retain their activity, i.e., while the expression of the negative regulators is still down regulated. In some embodiments, siRNAs capable of down regulating the expression of target genes are administered within 3 days after administration of siRNAs capable of down regulating the expression of negative regulators of RNAi. In yet other embodiments, siRNAs capable of down regulating the expression of negative regulators of RNAi are administered after siRNAs capable of down regulating the expression of target genes have been administered, and while they still retain their activity, i.e., while the expression of the target genes is still down regulated. In some embodiments, siRNAs capable of down regulating the expression of negative regulators of RNAi are administered within 3 days after administration of siRNAs capable of down regulating the expression of target genes.

As used herein, the term “retain their activity” means the administered nucleic acid molecules are not totally degraded and retain at least 10% of the maximum suppressive effects on the target genes; preferably, they retain at least 30% of the maximum suppressive effects on the target genes; more preferably, they retain at least 50% of the maximum suppressive effects on the target genes.

In some embodiments of the present invention, all or a portion of the siRNAs are chemically synthesized.

In some embodiments, all or a portion of the siRNAs are synthesized in vivo or in vitro using a nucleic acid sequence.

In some embodiments, the siRNAs are derived from precursor RNAs via chemical modification, biological modification, or a combination thereof.

As used herein, the term “chemically synthesized” means the siRNA molecules are synthesized using single nucleotides through a series of chemical reactions. Methods of synthesizing RNA molecules are known in the art. (See, for example, U.S. Pat. No. 7,056,704)

As used herein, the term “synthesized using a nucleic acid sequence” means that molecules that down regulate target RNA molecules are expressed from transcription units inserted into DNA or RNA vectors. The recombinant vectors are preferably DNA plasmids or viral vectors. For in vivo synthesis, the recombinant vectors capable of expressing the siRNA molecules are delivered as described herein, and persist in target subjects. Once expressed, the siRNA molecules bind to the target RNA and down-regulate its function or expression. Those skilled in the art realize that any nucleic acid can be expressed in eukaryotic cells from an appropriate DNA/RNA vector.

As used herein, the term “precursor RNA” refers to an RNA molecule from which siRNA molecules are derived, for example, by enzyme digestion, protecting group addition, and the like.

As used herein, the term “chemical modification” refers to any alteration of the RNA molecule by chemical reactions. For example, a 5′ and/or a 3′-cap structure can be added to protect the molecule from degradation in vivo. Preferably, such chemical modification does not significantly reduce the activity of siRNA molecules and does not have significant toxicity to the subject.

As used herein, the term “biological modification” refers to any alteration of RNA molecules by biological activities. For example, a long precursor RNA molecule can be digested by RNases, such as RNase III, to produce siRNA molecules.

In some embodiments, the disease which is treated by a method described herein is a cancer.

In some embodiments, the cancer is selected from the group consisting of pancreatic carcinoma, melanoma, colon carcinoma, lung carcinoma, kidney carcinoma, gastrointestinal stromal tumors (GIST), chronic myelomonocytic leukemia (CMML), acute myeloid leukemia (AML), chronic myeloid leukemia (CML), breast cancer, glioblastoma, ovarian carcinoma, endometrial carcinoma, hepatocellular carcinoma, renal cell carcinoma, thyroid carcinoma, lymphoid carcinoma, bladder carcinoma, prostate carcinoma, cervical carcinoma, non-Hodgkin lymphoma, oral cavity & pharynx carcinoma, head and neck cell carcinoma, stomach carcinoma, esophagus carcinoma, larynx carcinoma, brain & ONS carcinoma, liver & IBD carcinoma, ovary carcinoma, and nasopharyngeal carcinoma. Generally, an abnormal growth of tissue resulting from uncontrolled, progressive multiplication of cells and serving no physiological function is considered to be a cancer. Some embodiments of the methods described herein are effective in reducing and for eliminating cancers.

In a preferred embodiment, the cancer is a melanoma.

In some embodiments, the disease which is treated by the method of the present invention is a disease caused by a virus.

In some embodiments, the disease is selected from the group consisting of acquired immunodeficiency syndrome (AIDS), hepatitis A, hepatitis B, hepatitis C, hepatitis Delta, influenza, foot-and-mouth disease, dengue disease/hemorrhagic disease, measles/subacute sclerosing panencephalitis (SSPE), cephalitis and brain infection, glandular fever/chronic lymphocytic leukemia/lymphomas/nasopharyngeal carcinoma, adult T cell leukemia (ATL) and HTLV-I-associated myelopathy/tropical spastic paraparesis (HAM/TSP), a neurologic disease, cytomegalovirus inclusion disease/transplant arterial disease, sexually transmitted infection (STI), oral and cervical cancer/head and neck cancer/squamous cell carcinoma, fever blisters, genital sores, and a flu-like illness.

In a particularly preferred embodiment of the present invention, the disease is hepatitis B.

In some embodiments of the invention, the target gene is a gene associated with a disease, whose down regulation ameliorates the disease.

In some preferred embodiments of the invention, the target gene is a gene encoding a product selected from the group consisting of VEGF (vascular endothelial growth factor), VEGFR (vascular endothelial growth factor receptor), c-Raf(MAPKKK)/bcl-2, CEACAM6 (carcinoembryonic antigen-related cell adhesion molecule 6), EGFR (epidermal growth factor receptor), Bcr-abl, AML1/MTG8 (a chimeric transcription factor produced by t(8;21) chromosome translocation and causing AML), Btk (Bruton tyrosine kinase), LPA1 (lysophosphatidic acid), Csk (C-terminal Src kinase), PKC (protein kinase C)-theta, Bim1 (Bcl2-interacting mediator of cell death), P53 mutant, stat3 (signal transducer and activator of transcription 3), c-myc, SIRT1 [sirtuin (silent mating type information regulation 2 homolog) 1], ERK1, Cyclooxygenase-2, sphingosine 1-phosphate (SIP) receptor-1, insulin-like growth factor receptor, Bax, CXCR4 [chemokine (CXC motif) receptor 4], FAK (Focal adhesion kinase), EphA2 (erythropoietin related tyrosine kinase receptor 2), Matrix metalloproteinase, BRAF(V599E) (v-raf murine sarcoma viral oncoprotein homolog B1), Brk (breast tumor kinase), EBV(Epstein-Barr virus), FASE (fatty acid synthase), C-erbB-2/HER2 (human epidermal growth factor receptor 2), HPV (human papillomavirus) E6\E7, Livin/ML-LAP (melanoma inhibitor of apoptosis)/KIAP, MDR (multiple drug resistance), CDK-2 (cyclin dependent kinase 2), MDM-2 (murine double minute-2), PKC (protein kinase C)-α, TGF-β (transforming growth factor-β), H-Ras, K-Ras, PLK1 (Polo-like kinase), Telomerase, S100A10 (oncoprotein in colorectal cancer cells), NPM-ALK (nucleophosmin-anaplastic lymphoma kinase), Nox1 (NADPH oxidase homolog 1), Cyclin E, Gp210 (pore membrane glycoprotein), c-Kit, survivin, Philadelphia chromosome, ribonucleotide reductase, Rho C, ATF2 (activating transcription factor 2), P110a, P10B of PI 3 kinase, Wt1 (Wilms' tumor), Pax2 (oncoprotein in human breast cancer), Wnt4, beta-catanin, integrin, urokinase-type plasminogen activator, Hec1 (highly expressed in cancer), Cyclophilin A, DNMT (DNA methyltransferase), MUC1 (mucin 1, transmembrane), Acetyl-CoA Carboxylase {alpha}, Mirk (Minibrain-related kinase)/Dyrk1b, MTA1 (metastasis-associated gene 1), SMYD3 (histone methyltransferase), ACTR (also called AIB1 and SRC-3, a coactivator for nuclear receptors), Hath1 (oncoprotein in colon adenocarcinomas), Mad2 (oncoprotein in ovarian cancer), STK15 (also known as BTAK and aurora2, a centrosome-associated kinase), XIAP (x-linked inhibitor of apoptosis, chemoresistance of pancreatic carcinoma cell), CD147/EMMPRIN (extracelluar matrix metalloproteinase inducer), ENPP2 [ectonucleotide pyrophosphatase/phosphodiesterase 2 (autotaxin)]/ATX /ATX-X/FLJ26803/LysoPLD/NPP2/PD-IALPHA/PDNP2, AKT (protein kinase B), PrPC (cellular prion protein, glycosylphosphatidylinositol-anchored membrane protein), Thioredoxin reductase 1, HSPG2 (heparan sulfate proteoglycan 2/perlecan), p38 MAP (mitogen-activated protein) kinase, hTERT (human telomerase reverse transcriptase), alphaB-Crystallin (a novel oncoprotein that predicts poor clinical outcome in breast cancer), STAT6 (signal transducer and activator of transcription 6), choline kinase, cyclin D1/CDK4, ASH1 (absent, small, or homeotic discs 1 as function histone methyltransferase activity), osteopontin (overexpression in laryngeal squamous cell carcinomas), 3-alkyladenine-DNA glycosylase, Plasmalemmal vesicle associated protein-1, SHP2 (a Src homology 2-containing tyrosine phosphatase), STAT5 (signal transducer and activator of transcription 5), Gab2 (GRB2-associated binding protein 2, a pivotal role in the EGF-induced ERK activation pathway), Etk/BMX (a non-receptor protein tyrosine kinase), AFP (alpha-fetoprotein), Id1/Id3 gene (up-regulated in papillary and medullary thyroid cancers), Maternal embryonic leucine zipper kinase/murine protein serine-threonine kinase 38, phosphatidylethanolamine-binding protein 4, ATP citrate lyase, cyclophilin A, DNA-PK (DNA-dependent protein kinase), CT120A (a new gene of lung cancer), EBNA1 (Epstein-Barr nuclear antigen 1), Pim family kinases, hypoxia-inducible factor-1alpha, acetyl-CoA-carboxylase-alpha, Rac 1/RAC3, Aurora-B (previously known as AIM-1, a conserved eukaryotic mitotic protein kinase, overexpressed in various cancer cells), platelet-derived growth factor-D/platelet-derived growth factor receptor beta, Androgen Receptor, EN2 (a candidate oncoprotein in human breast cancer), Vav1 (a signal transducing protein required for T cell receptor (TCR) signals that drive positive and negative selection in the thymus), BRCA1 (a breast cancer susceptibility gene), the nonreceptor protein-tyrosine kinase Pyk2 (proline-rich tyrosine kinase 2), leptin, hLRH-1 (human nuclear receptor 1), p28GANK (oncoprotein in Hepatocellular Carcinoma), MCT-1 (a novel candidate oncoprotein with homology to a protein-protein binding domain of cyclin H), Fibroblast growth factor receptor 3, p53R2 [ribonucleotide reductase (RR)], integrin-linked kinase, cdc42 (cell division cycle 42), MAT2A (oncogene in hepatoma cells), intercellular adhesion molecules (ICAMs), mimitin (cell proliferation of esophageal squamous cell carcinoma), RET (proto-oncogene, a segment of DNA that provides the code that cells in the body use to produce a structure called a membrane receptor), S-phase kinase-interacting protein 2, NRAS (neuroblastoma RAS viral (v-ras) oncogene homolog), phosphatidylinositol 3-kinase, Fas-ligand, IGFBP-5 (insulin-like growth factor-binding protein-5), E2F4 (E2F transcription factor 4), FLT3 (fms-related tyrosine kinase 3), estrogen receptor, LYN kinase (overexpression in chronic myelogenous leukemia cells), cathepsin B, ZNRD1 (a new zinc ribbon gene has been previously identified as an upregulated gene in a multidrugresistant gastric cancer), ARA55 (androgen receptor coregulator), and activin.

In a preferred embodiment, the target gene is c-myc gene.

In some embodiments, the target gene is a viral gene.

In some embodiments, the target gene is a gene of a virus selected from a group consisting of human immunodeficiency virus, hepatitis A virus, hepatitis B virus, hepatitis C virus, hepatitis delta virus, influenza virus, foot-and-mouth disease virus, dengue virus type 2, measles virus, panencephalitis virus, Epstein-Barr virus, human T-cell leukemia virus, Measles virus, cytomegalovirus, human papillomavirus, and herpes simplex virus.

In a preferred embodiment, the target gene is a gene encoding polymerase of hepatitis B virus.

As used herein, the term “a gene associated with a disease” refers to a gene whose abnormal expression causes a disease or contributes to the development of a disease. Alternatively, a gene whose normal expression may also cause a disease or contribute to the development of a disease under certain circumstances is also a gene associated with a disease.

As used herein, the term “viral gene” refers to a gene encoded by a virus, whose abnormal expression kills the virus or inhibits replication of the virus.

In a preferred embodiment of the invention, the target gene is a c-myc gene and the negative regulator of RNAi is THEX1, ADAR1, or a combination thereof.

In another preferred embodiment, the target gene is a gene encoding polymerase of hepatitis B virus and the negative regulator of RNAi is THEX1.

In some embodiments, compositions are provided that include one or more siRNAs, or precursors thereof, capable of down regulating the expression of one or more target genes and comprising one or more siRNAs, or precursors thereof, capable of down regulating the expression of one or more negative regulators of RNAi.

As used herein, the term “composition” refers to a mixture which includes a pharmaceutically effective amount of the desired siRNA in a pharmaceutically acceptable carrier or diluent. The composition should be in a form suitable for administration, e.g., systemic administration, into a cell or subject, preferably a human. Suitable forms, in part, depend upon the use or the route of entry, for example oral, transdermal, or by injection. Such forms should not prevent the composition from reaching a target cell (i.e., a cell to which the siRNA is desired to be delivered to). For example, compositions injected into the blood stream should be soluble. Acceptable carriers or diluents for therapeutic use are well known in the pharmaceutical art. Other factors are also known in the art, and include considerations such as toxicity and forms which prevent the composition or formulation from exerting its effect.

A pharmaceutically effective amount is that amount required to prevent, delay, inhibit the occurrence, or treat (alleviate a symptom to some extent, preferably all of the symptoms) of a disease state. The pharmaceutically effective amount depends on the type of disease, the composition used, the route of administration, the type of subject being treated, the physical characteristics of the specific subject under consideration, concurrent medication, and other factors which those skilled in the medical arts will recognize.

The siRNAs can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like.

siRNAs may be expressed in vivo or in vitro from nucleotide sequences before they exhibit their functions. Therefore, in some embodiments, a composition is provided that includes one or more nucleotide sequences encoding one or more siRNAs, or precursors thereof, capable of down regulating the expression of one or more target genes, and also includes one or more siRNAs, or precursors thereof, capable of down regulating the expression of one or more negative regulators of RNAi, is provided.

In another embodiment, a composition is provided that includes one or more siRNAs, or precursors thereof, capable of down regulating the expression of one or more target genes, and also includes one or more nucleotide sequences encoding one or more siRNAs, or precursors thereof, capable of down regulating the expression of one or more negative regulators of RNAi.

In still another embodiment, a composition is provided that includes one or more nucleotide sequences encoding one or more nucleotide sequences encoding one or more siRNAs, or precursors thereof, capable of down regulating the expression of one or more target genes, and also includes one or more nucleotide sequences encoding one or more siRNAs, or precursors thereof, capable of down regulating the expression of one or more negative regulators of RNAi.

In some embodiments, the ratio of siRNAs capable of down regulating the expression of target genes to siRNAs capable of down regulating the expression of negative regulators of RNAi in the composition is in a range of about 5:1 to about 20:1 (w/w). In a preferred embodiment of the invention, the ratio of siRNAs capable of down regulating the expression of target genes to siRNAs capable of down regulating the expression of negative regulators of RNAi in the composition is about 10:1 (w/w).

In some embodiments, methods are provided for determining the optimal ratio of siRNAs capable of down regulating the expression of one or more target genes to siRNAs capable of down regulating the expression of one or more negative regulators of RNAi, e.g., for use in a method for enhancing siRNA efficacy as described herein. The methods can include the following steps, in any order:
(a) inducing expression of genes encoding a negative regulator of RNAi using any siRNA molecules;
(b) determining an effective dose of siRNA molecules that is able to induce high expression of the negative regulators of RNAi;
(c) based on the high expression of the negative regulators of RNAi determined in (b), determining a dose of an siRNA that down regulates expression of the negative regulator of RNAi to a base expression level; and
(d) based on the down regulation of the negative regulator of RNAi determined in (c), determining the dose of an siRNA that down regulates expression of one or more target genes.

As used herein, the term “high expression” means that the negative regulators of RNAi are expressed at a level such that the suppression rate of siRNA targeting a gene is below 30%, preferably below 20%, most preferably below 10% of the optimal level of suppression in the absence of expression of the negative regulators.

As used herein, the term “base expression level” means that the negative regulators of RNAi are expressed at a level as if there were no siRNA molecules in the cell.

In some embodiments, an effective dose of siRNA molecules that is able to induce high expression of negative regulators of RNAi refers to a dose at which the siRNA molecules are administered so as to result in an increase of at least 2-fold of the expression level of a negative regulator of RNAi, as determined by RT-PCR. The dose of an siRNA that down regulates expression of a negative regulator of RNAi to base expression levels is determined by administration of different amounts of the siRNA. RT-PCR can be used to determine the expression level of the negative regulator of RNAi. Based on the amount of siRNA that down regulates expression of the negative regulator of RNAi to base expression levels, the dose of the siRNA that down regulates expression of a target gene to the lowest level can be determined. The lowest level of target gene expression means more siRNA cannot significantly cause further reduction of the mRNA level of the target gene.

Other features and advantages of the invention will be apparent from the following description of the working examples, and from the claims. The following working examples are provided by way of illustration and are not intended to limit the present invention.

### EXAMPLES

Unless specified otherwise, all of the chemical reagents used in the working examples were purchased from TakaRa, Japan.

### Methodology

### Preparation of siRNA

Large scale dsRNA and siRNA molecules used in RNAi were obtained by using the method comprising the steps of:
(I) construction of plamid vectors expressing dsRNA with stem loop structure;
(II) E. coli transformation;
(III) fermentation of E. coli;
(IV) extraction of total RNA and plasmid DNA by alkali-SDS extraction;
(V) purification of dsRNA by CF-11 column; and
(VI) processing of the dsRNA molecules into siRNA molecules with the length of 20-30 bp by E. coli RNase III or animal or plant dicer enzymes.

### Construction of Plamid Vectors Expressing dsRNA with Stem Loop Structure

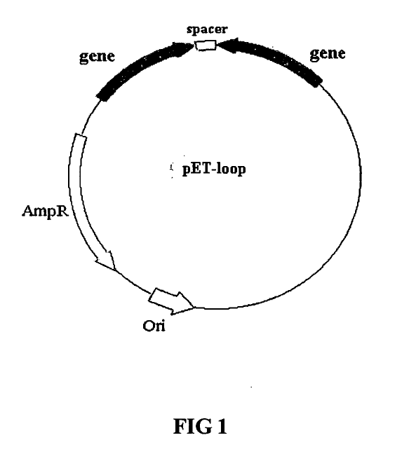

The map of the plamid vector is shown in FIG. 1. Specifically, the plasmid vector, pET-loop, was constructed by inserting about 300 bp DNA fragment obtained from yeast or any other organism than E. coli, into pET-22b vector (Novagen, Madison Wis.) between BamHI and EcoRI sites.

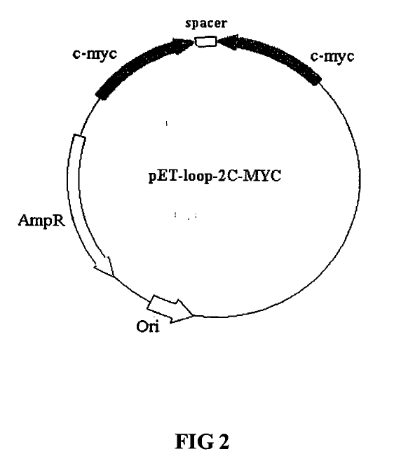

To obtain a plasmid vector from which precursor dsRNA of the siRNA targeting the mouse c-myc gene can be expressed, the DNA fragment containing the coding region of the mouse c-myc gene was PCR-amplified from mouse cDNA (Invitrogen, USA) with the following primers: c-myc-sense: 5′-GCGGGTACCCTGTTTGAAGGCTGGATTT-3′ (SEQ ID NO:1, the introduced EcoRI site is underlined) and c-myc-antisense: 5′-ATGCGAATTCTACAGGCTGGAGGTGGAGCA-3′ (SEQ ID NO:2, the introduced KpnI site is underlined). The PCR program was 94° C. for 1 minute, 52° C. for 0.5 minutes and 72° C. for 1 minute, with 30 cycles. The obtained DNA fragment was first cloned into pBluescript II KS (Stratagene) with the introduced restriction enzyme recognition sites and sequence-verified, then subcloned into pET-loop between the EcoRI and KpnI sites to obtain the plasmid pET-loop-2C-MYC (FIG. 2).

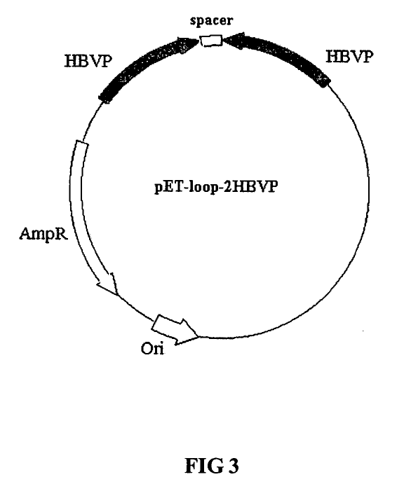

To obtain a plasmid vector from which precursor dsRNA of siRNA targeting the gene encoding the polymerase of hepatitis B virus can be expressed, the DNA fragment containing the coding region of the gene was PCR-amplified from the hepatitis B virus genome DNA with the primers of HBVP-sense: 5′-GGAATTCGTCTTGGGTATACATTTGACC-3′ (SEQ ID NO:3, the EcoRI recognition site is underlined) and HBVP-antisense: 5′-GGGGTACCAGAGGACAACAGAGTTG-3′ (SEQ ID NO:4, the KpnI recognition site is underlined) under the same PCR condition as described above. The obtained DNA fragment was inserted into pET-loop as described above and the plasmid pET-loop-2HBVP was obtained (FIG. 3).

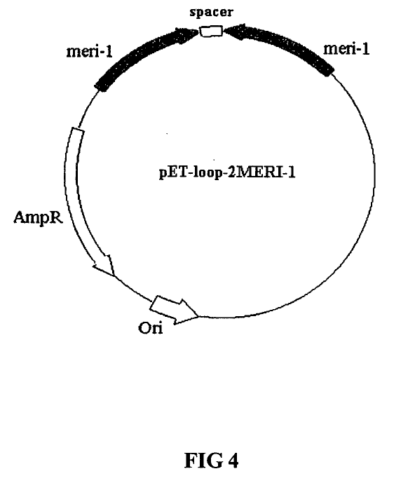

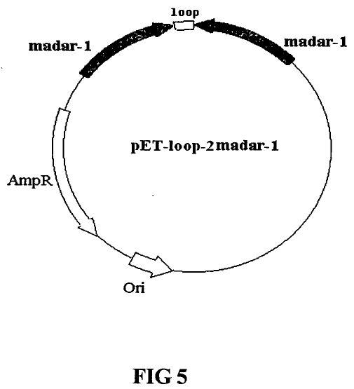

To obtain a plasmid vector from which precursor dsRNA of siRNA targeting the mouse thex1 gene (esiMERI-1) can be expressed, the DNA fragment containing exon 2 and exon 3 of seven mouse eri-1 exons (MERI-1) (GenBank® accession number NM_{—}026067) was PCR-amplified from mouse cDNA with the primers of eri-1-sense 5′-CGGAATTCGCAGACTTGAT-3′ (SEQ ID NO:5, the introduced EcoRI site is underlined) and eri-1-antisense 5′-CCGGTACCTGGCCTCACATA-3′ (SEQ ID NO:6, the introduced KpnI site is underlined) under the same PCR condition as described above. The obtained DNA fragment was first cloned into pUC118 (Stratagene) with the introduced restriction enzyme recognition sites and sequence-verified, then subcloned into pET-loop between the EcoRI and KpnI sites to obtain the plasmid pET-loop-2MERI-1 (FIG. 4).

To obtain a plasmid vector from which precursor dsRNA of siRNA targeting the mouse adar-1 gene (esiMADAR-1) can be expressed, the DNA fragment containing exon 5 and exon 6 of fifteen mouse adar-1 exons (MADAR-1) (GenBank® accession number AY488122) was amplified from cDNA with the following primers: adar-1-sense 5′-GCGAATTCGTTCCAGTACTGTGTAGCAGT-3′(SEQ ID NO:7, the introduced EcoRI site is underlined) and adr-1-antisense 5′-ATGCGGTACCGGATCCTTGGGTTCGTGAGGAGGTCC-3′ (SEQ ID NO:8, the introduced KpnI site is underlined) under the same PCR conditions as described above. The obtained DNA fragment was first cloned into pBluescript II KS (Stratagene) with the introduced restriction enzyme recognition sites and sequence-verified, then subcloned into the pET-loop between the EcoRI and KpnI sites to obtain the plasmid pET-loop-2MADAR-1. (FIG. 5)

To obtain a plasmid vector from which precursor dsRNA of siRNA targeting the avian influenza virus NP gene (esiNP) can be expressed, the DNA fragment encoding part of the avian influenza virus NP (nucleoprotein) was amplified from cDNA with following primers: np-sense 5′-GCGAATTCTCTGCACTCATCCTGAGAGG-3′ (SEQ ID NO:9, the introduced EcoRI site is underlined) and np-antisense 5′-CGGGTACCTACTCCTCTGCATTGTCTCC-3′(SEQ ID NO: 10, the introduced KpnI site is underlined) under the same PCR condition as described above. The obtained DNA fragment was first cloned into pBluescript II KS (Stratagene) with the introduced restriction enzyme recognition sites and sequence-verified, then subcloned into pET-loop between the EcoRI and KpnI sites to obtain the plasmid pET-loop-2NP.

### E. coli Transformation

The dsRNA expression vectors obtained above were transformed into E. coli strain BL21(DE3) (Stratagene) as described in Qian et al., World J. Gastroenterol. 11:1297-302 (2005).

### Fermentation of E. coli

After transformation, BL21 (DE3) strains containing the dsRNA expression vectors were inoculated into 200 ml of LB (Luria-Bertani) medium supplemented with 100 μg/ml ampicillin and cultured with shaking (250 rev./minute) at 37° C. overnight. The culture was then inoculated into a small fermentation tank containing 25 L of fresh medium and continued growing 8-9 hours before inoculation into a large fermentation tank (vol. 500 L) containing 300 L of fresh medium. The E. coli was further fermented in the large tank for 3 hours at 37° C. before 6 kg lactose was added into the culture to induce the expression of dsRNA. Then the E. coli was further fermented for 3 hours and the cells were harvested by centrifugation at 3800 g for 15 minutes (Model GL105, Shanghai Centrifuge Institute Co., LTD.).

### Extraction of Total RNA and Plasmid DNA by Alkali-SDS Extraction

One hundred grams of the E. coli cells were suspended in 1000 ml suspension buffer (50 mM Glucose, 25 mM Tris-HCl and 10 mM EDTA pH 8.0). Two liter of lysis buffer (0.2 M NaOH and 2% SDS) was added, then 1500 ml solution of potassium acetate was added after a gentle stir. The solution was stirred gently again, and the total solution was divided into several flasks and ice-cooled for 10 minutes. A centrifugaion of 10 minutes was performed at 10000 g (J-6B centrifuge, Beckman), then the supernatant was collected and mixed with equal volume of phenol-chloroform-isoamyl alcohol (25:24:1). The mixture was mixed well by vortex and was centrifuged for 10 minutes at 10000 g(J-6B centrifuge, Beckman). The supernatant was collected for future use.

### Purification of dsRNA by CF-11 Column

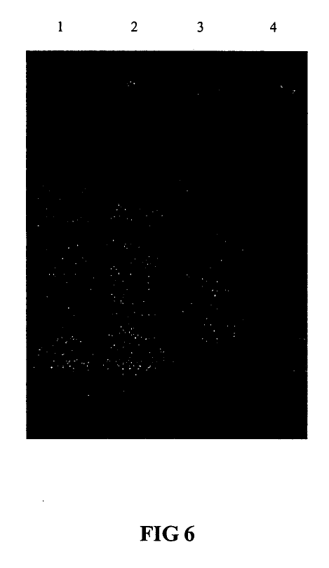

The dsRNA purification and esiRNA (Escherichia-coli-expressed and enzyme-digested siRNAs) preparation were performed using the method described in Mulkeen et al., J. Surg. Res. 121:279-280 (2004). Briefly, the RNA-containing cell lysate obtained as described above was diluted in ethanol to a final concentration of 20%, then the solution was passed through a Whatman® fibrous cellulose CF-11 column (Whatman, USA) equilibrated with 20% ethanol containing 1×STE (10 mM Tris/HCl, 100 mM NaCl and 1 mM EDTA, pH 8.0). The column was stored at 4° C. and the column-purification was performed at 4° C. after the sample had been placed on ice for 10 minutes. After washing with 5 L of 1×STE containing 17% ethanol, dsRNA was then eluted out of the column with 2 L of 1×STE which was pre-heated to 55° C. FIG. 6 shows the electrophoresis result of the unpurified and purified samples (lanes 1 and 3, respectively) in the agarose gel. Compared with normal plasmids (lanes 2 and 4), the purified sample (lane 3) is long dsRNA.

### Processing of the dsRNA Molecules into siRNA Molecules

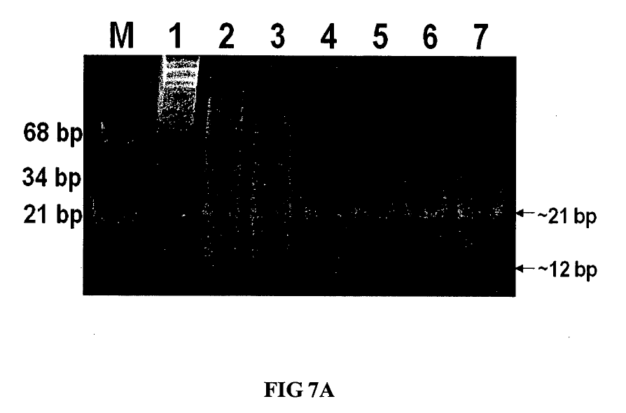

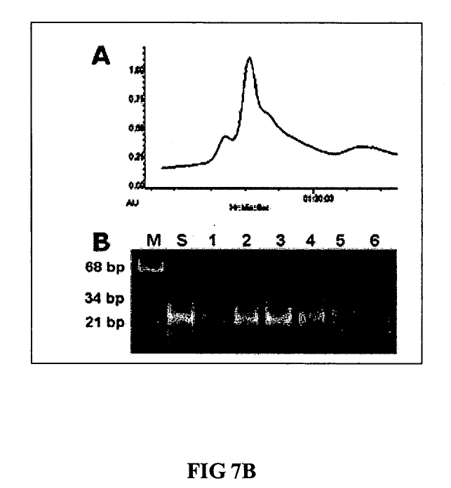

To prepare siRNAs, every 4 μg of purified long dsRNA was digested with 0.1 μg of recombinant RNase III (Ambion) in a reaction mixture containing 50 mM Tris/HCl (pH 7.5), 50 mM NaCl, 10 mM MnCl₂ and 1 mM DTT at 37° C. for 1 hour. The digestion mixture was separated on a 15% non-denaturing polyacrylamide gel, and the result is shown in FIG. 7A. The digested products were further purified on Superdex-75 column (Pharmacia/Amersham, USA) to obtain pure 21-23 bp esiRNA as shown in FIG. 7B.

### Construction of Reporter Plasmid pCMV-iHBS

Reporter plasmid pCMV-iHBS was constructed as described in Xu et al., Biochem. Biophys. Res. Commun. 329:538-543 (2005), containing an HBsAg (type B hepatitis virus surface antigen)-coding sequence placed downstream of mouse Igκ-chain leader sequence, which enables the expressed protein to secrete to the outside the cell. The secretory plasmid was used for both cell culture assay and animal testing.

### Cell Culture and Transfection

CHO (Chinese-hamster ovary) cells (ATCC) were grown at 37° C. in an atmosphere of 5% CO₂ in Dulbecco's modified Eagle's medium supplemented with 10% fetal calf serum (Biological Industries, Kibutz Beit Haemek, Israel), streptomycin (100 μg/ml) and penicillin (100 units/ml). To establish a cell line that constitutively expresses HBsAg, 600 ng/ml pCMV-iHBS plasmid DNA was transfected into CHO cells in a 24-well plate (70% confluence) using Lipofectamine™ 2000 (Invitrogen), according to the manufacturer's instructions. The level of HBsAg in the medium was measured 72 hours after transfection, and G418 was added to cells to a concentration of 800 μg/ml. G418-resistant cells were then serially diluted to make constitutively expressive clonal HBsAg strains named CHO-iHBS cell strains.

### Administration of siRNAs to Mice

A solution containing siRNA (siRNA 5-30 μg, NaCl 8.6 g, KCl 0.3 g, and CaCl₂ 0.13 g in a total volume of 1000 ml water solution) was administered to male ICR mice (6-8 weeks old, 18-20 g; Shanghai Laboratory Animal Center, Shanghai, China) by intraperitoneal injection or by hydrodynamic injection (high-volume intravenous injection) at a dose of 1-30 μg/kg body weight. Control mice were injected with the same solution without siRNA. The procedure was performed in accordance with the requirements of the Shanghai Laboratory Animal Center of Shanghai, which proved the procedure to be safe for animals.

### RT (Reverse Transcription)-PCR Analysis

Total RNA was isolated from freshly harvested livers of mice injected with pCMV-iHBS plasmid DNA and siRNAs using a Qiagen RNA isolation kit (Qiagen, Germany). RT was performed from total RNA using RNase-free MMLV (Moloney murine leukemia virus) reverse transcriptase (Takara, Osaka, Japan). To correct the amplification process for tube-to-tube variability in amplification efficiency, β-actin mRNA was used as an internal standard for the semiquantification of the RT-PCR. The primers for β-actin were 5′-TGATGGACTCCGGTGACGG-3′(SEQ ID NO:11, forward) and 5′-TGTCACGCACGATTTCCCGC-3′ (SEQ ID NO:12, reverse). After normalization with β-actin amplicon (179 bp), the same amount of cDNA was used as a template to amplify thex-1 and adar-1 genes using the following primers: thex-1-sense primer 5′-CGGAATTCGCAGACTTGAT-3′ (SEQ ID NO:13) and thex-1-antisense primer 5′-CCGGTACCTGGCCTCACATA-3′ (SEQ ID NO:14); adar-1-sense primer 5′-GCTCTAGAGTTCCAGTACTGTGTAGCAGT-3′(SEQ ID NO:15) and adar-1-antisense primer 5′-ATGCGAATTCGGATCCTTGGGTTCGTGAGGAGGTCC-3′ (SEQ ID NO:16). The PCR program was set up as follows: denaturing at 94° C. for 1 minute, annealing at 52° C. for 0.5 minutes and extension at 72° C. for 1 minute. The number of amplification cycles was 30 for thex-1 and adar-1 genes and 25 for β-actin. Then 15, 20, 25, 30, 35, 37 and 40 cycles of each kind of RT-PCR were performed to verify that under the described conditions the PCR-amplification of each fragment was still in the linear range. Samples were analysed on a 2% agarose gel stained with ethidium bromide. The density of bands was quantified by using a Molecular Imager FX Pro Fluorescent Imager (Bio-Rad).

### Example 1

### Higher Doses of siRNA Induced Stronger Rebound of HBsAg Expression after a Period of Suppression in CHO-iHBS Cells

For esiRNA dose-response experiments, CHO-iHBS cells from six-well plates (70% confluence, approx. 5×10⁶ cells) were transfected with 4-10 μg of esiHBVP using Gene Pulser Xcell™ system (Bio-Rad) according to the manufacturer's instructions. Cells were immediately seeded into new six-well plates with fresh medium. Every 24 hours, medium was removed for analysis, and the cells were replenished with fresh medium. Secretory HBsAg in the medium was analysed using an ELISA.

It is known in the art that the down regulation of gene expression is sequence-specific and dose-dependent, and that the RNAi effect is transient and usually lasts 3-4 days (Xuan et al., Mol. Biotechnol. 203-209 (2005)). It has been suggested that the expression of homologous genes rebound after 3-4 days of suppression by siRNAs and that the rebound effect is stronger in cells or animals challenged with higher doses of siRNAs than in those challenged by lower doses of siRNAs.

To examine suppressive effects of esiRNA on hepatitis B virus polymerase (HBVP), CHO-iHBS cells were transfected with 4 μg or 10 μg of esiHBVP dissolved in PBS. Approximately 5×10⁶ cells/well were used for transfection and the same volume of PBS without any DNA was used as a negative control. The concentration of HBsAg secreted into the medium at various time points after transfection was measured and the expression of secretory HbsAg was normalized relative to the negative control.

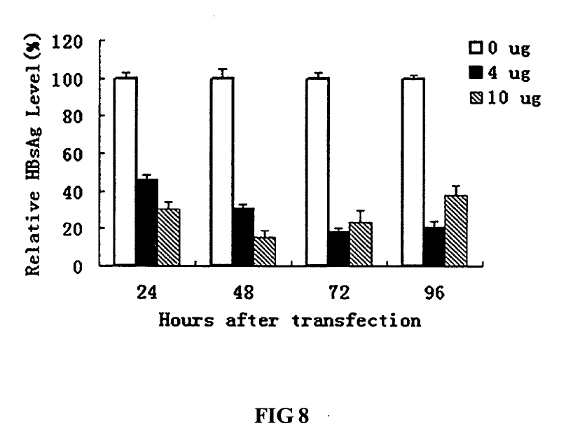

The results showed a continuous increase of suppression of HBsAg expression in cells transfected with 4 μg of esiHBVP from 24 to 72 hours before a slight rebound at 96 hours post-transfection, while cells given 10 μg of esiHBVP elicited a better suppressive effect at an earlier stage and began to rebound at 72 hours post-transfection (FIG. 8). It seemed that the suppressive effect of RNAi began to be lost at later time points and the overall expression level of the gene in the cells began to rise. Interestingly, the cells given higher doses of siRNA showed a much higher rebound at 96 hours after transfection. To explain this phenomenon, it might be possible that some sort of repelling mechanism was triggered in the cell when large amounts of siRNA were introduced into cells, to protect cells from RNA viral infection.

### Example 2

### Higher Doses of siRNA Induced Stronger Rebound of HBsAg Expression after a Period of Suppression in Mice

The stronger rebound of HBsAg expression induced by higher doses of siRNA described in cells in Example 1 was also observed in animals.

E. coli-expressed siRNA targeting the gene encoding the polymerase of hepatitis B virus (esiHBVP) (1 μg or 10 μg) and 10 μg pCMV-iHBS were injected into mice by hydrodynamic injection. Only 10 μg pCMV-iHBS was injected into control mice. The surface antigen of the hepatitis B virus (HbsAg) in serum was measured using the ELISA at different time points 24 hours after injection.

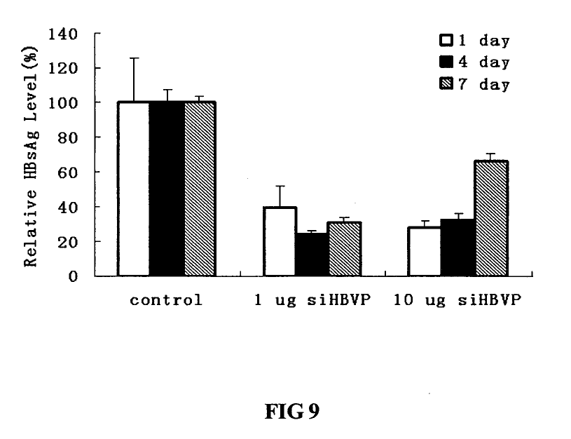

As shown in FIG. 9, in the control group, HbsAg concentration in serum reached the highest level at 24 hours after injection, and remained stable for 7 days. Injection of esiHBVP started to suppress the expression of HbsAg on the first day after injection, and the suppression was dose-dependent (60% and 70% suppression by 1 μg and 10 μg esiHBVP, respectively). On day 4, the suppression rate by 1 μg esiHBVP reached 88%, however, the suppression rate by 10 μg esiHBVP decreased to 42%. On day 7, the suppression rate by 1 μg esiHBVP still remained at 70%, but the suppression rate by 10 μg esiHBVP had decreased to 30%.

### Example 3

### RT-PCR Analysis of the Expression Levels of eri-1 Gene in Mice (thex-1)

It was theorized that a stronger rebound of HBsAg expression induced by higher doses of siRNA in both a cell line and in animals was due to the high dose esiHBVP (10 μg) molecules up-regulating the expression of negative regulators of RNAi, such as THEX1 and ADAR1. It was then examined whether or not the expression level of thex1 or adar-1 in the liver changed when siRNA was introduced into the body.

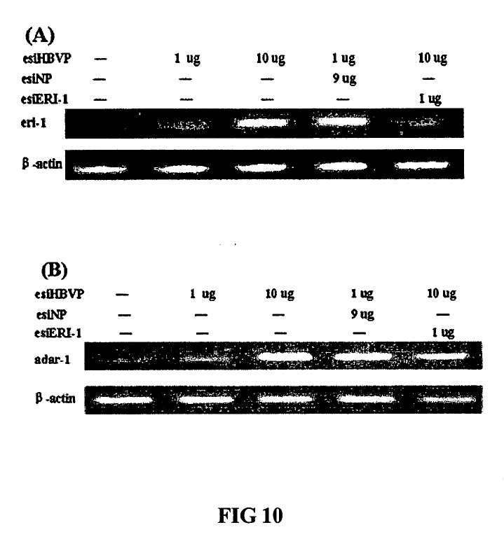

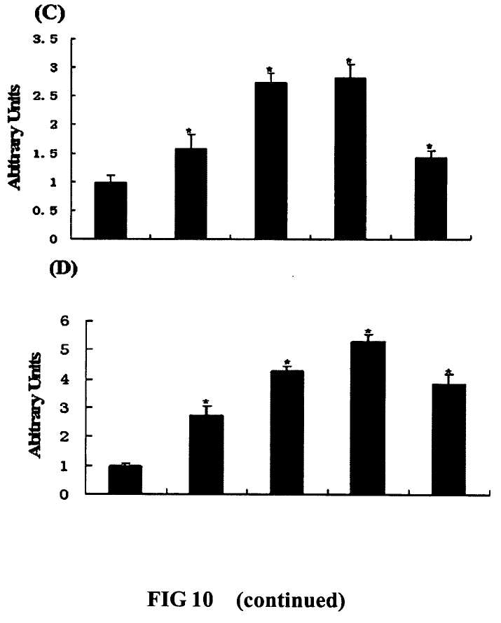

Various amounts of esiHBVP or non-related control esiNP were injected into mice by hydrodynamic injection. At 4 days after administration, total RNA was extracted from the animals' livers and RT-PCR was performed using thex-1 and adar-1 gene-specific primers. All reactions were normalized with β-actin. As shown in FIGS. 10A-D, the mRNA levels of thex-1 and adar-1 genes were increased markedly by the introduction of exogenous siRNAs. The group injected with 10 μg of esiHBVP showed a near 3-fold increase of mRNA level with the thex-1 gene and over 4-fold increase with the adar-1 gene than the uninjected group. The increase was also observed in the group injected with 1 μg esiHBVP plus 9 μg of non-specific esiNP. However, when 1 μg of esiERI-1 was injected into mice together with 10 μg of esiHBVP, the mRNA levels of both thex-1 and adar-1 were reduced. In particular, the thex-1 mRNA showed a level close to that of mice injected with only 1 μg of esiHBVP. Therefore, the administration of high doses of exogenous siRNAs, either 10 μg of esiHBVP or 1 μg of esiHBVP plus 9 μg of esiNP, induced the expression of thex-1 and adar-1 genes, and the addition of 1 μg of esiERI-1 offset, to some extent, the increase of thex-1 mRNA.

### Example 4

### Silencing of eri-1 Homolog Gene (thex-1) make a RNAi more Effective in Mouse Liver

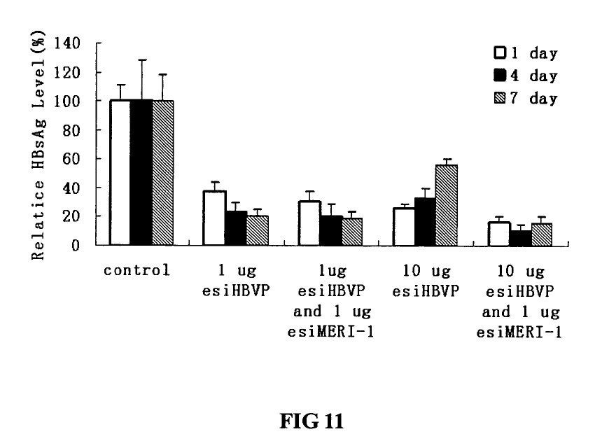

In a similar experiment to the one described in Example 3, 1 μg siRNA targeting mouse thex1 gene (esiMERI-1) was co-administered with 1 μg and 10 μg esiHBVP. As shown in FIG. 11, at day 4 and day 7 after injection, suppression of HBsAg expression by 10 μg esiHBVP still remained at high level and the suppression by esiHBVP was in a dose-dependent manner. Meantime, suppression of HBsAg expression by 1 μg esiHBVP was also improved. These results demonstrated that down regulation of thex1 gene results in significant improvement of RNAi.

### Example 5

### Inhibition of Melanoma B16 Cell Growth in Mice by siRNA Targeting the c-myc Gene and siRNA Targeting the thex1 Gene and the adar-1 Gene

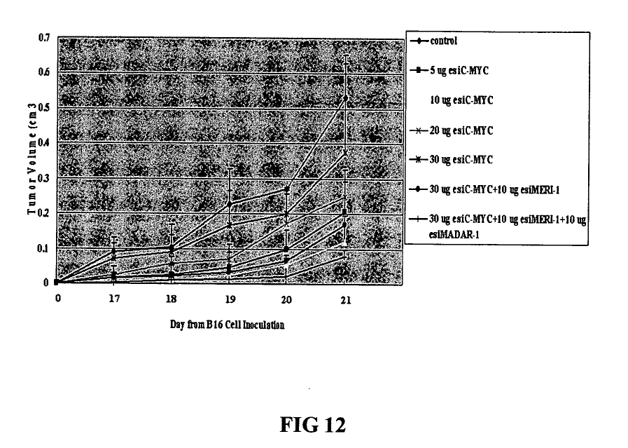

siRNA targeting mouse c-myc gene (esiC-MYC) was injected intraperitoneally into melanoma-bearing mice as described above. The doses of the siRNAs are indicated in FIG. 12. The injection was performed once a day within a period of 20 days and the tumor volume was recorded in FIG. 12. Compared to the control group, injection of 5 μg esiC-MYC and 10 μg esiC-MYC siRNA inhibited the growth of mouse melanoma in a dose-dependent manner (80% and 88% inhibited, respectively). However, injection of 20 μg esiC-MYC and 30 μg esiC-MYC siRNA inhibited the growth of mouse melanoma less efficiently (80% and 60% inhibited, respectively). These results are consistent with the hypothesis that high dose siRNA molecules up-regulate the expression of thex1 gene and adar-1 gene, and that part of the siRNA molecules were subsequently degraded by THEX1.

When 10 μg siRNA targeting mouse thex gene (esiMERI-1) and 10 μg siRNA targeting mouse adar-1 gene (esiMADAR-1) were co-administered with 30 μg esiC-MYC, the tumor growth was inhibited even more significantly (98%), as shown in FIG. 12.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a disease or a disorder, the method comprising administering to a subject (i) one or more siRNAs capable of down regulating expression of one or more target genes, and (ii) one or more siRNAs capable of down regulating expression of one or more negative regulators of RNAi.

2. The method of claim 1, wherein the ratio of the siRNAs capable of down regulating expression of target genes to the siRNAs capable of down regulating expression of negative regulators of RNAi is in a range of about 5:1 to about 20:1 (w/w).

3. The method of claim 2, wherein the ratio of the siRNAs capable of down regulating expression of target genes to the siRNAs capable of down regulating expression of negative regulators of RNAi is about 10:1 (w/w).

4. The method of claim 1, wherein the siRNAs capable of down regulating expression of target genes and the siRNAs capable of down regulating expression of negative regulators of RNAi are administered at the same time.

5. The method of claim 1, wherein the siRNAs capable of down regulating expression of target genes are administered after the siRNAs capable of down regulating expression of negative regulators of RNAi have been administered, and still retain their activity.

6. The method of claim 5, wherein the siRNAs capable of down regulating expression of target genes are administered within 3 days after administration of the siRNAs capable of down regulating expression of negative regulators of RNAi.

7. The method of claim 1, wherein the siRNAs capable of down regulating expression of negative regulators of RNAi are administered after the siRNAs capable of down regulating expression of target genes have been administered, and still retain their activity.

8. The method of claim 7, wherein the siRNAs capable of down regulating expression of negative regulators of RNAi are administered within 3 days after administration of the siRNAs capable of down regulating expression of target genes.

9. The method of claim 1, wherein all or a portion of the siRNAs are chemically synthesized.

10. The method of claim 1, wherein all or a portion of the siRNAs are synthesized in vivo or in vitro using a nucleic acid sequence.

11. The method of claim 1, wherein all or a portion of the siRNAs are derived in vivo or in vitro from precursor RNAs via chemical modification, biological modification or combinations thereof.

12. The method of claim 1, wherein the disease is a cancer.

13. The method of claim 12, wherein the cancer is selected from the group consisting of pancreatic carcinoma, melanoma, colon carcinoma, lung carcinoma, kidney carcinoma, gastrointestinal stromal tumors (GIST), chronic myelomonocytic leukemia (CMML), acute myeloid leukemia (AML), chronic myeloid leukemia (CML), breast cancer, glioblastoma, ovarian carcinoma, endometrial carcinoma, hepatocellular carcinoma, renal cell carcinoma, thyroid carcinoma, lymphoid carcinoma, bladder carcinoma, prostate carcinoma, cervical carcinoma, non-Hodgkin lymphoma, oral cavity & pharynx carcinoma, head and neck cell carcinoma, stomach carcinoma, esophagus carcinoma, larynx carcinoma, brain & ONS carcinoma, liver & IBD carcinoma, ovarian carcinoma, and nasopharyngeal carcinoma.

14. The method of claim 13, wherein the cancer is a melanoma.

15. The method of claim 1, wherein the disease is a disease caused by a virus.

16. The method of claim 15, wherein the disease is selected from the group consisting of acquired immunodeficiency syndrome (AIDS), hepatitis A, hepatitis B, hepatitis C, hepatitis Delta, influenza, foot-and-mouth disease, dengue disease/hemorrhagic disease, measles/subacute sclerosing panencephalitis (SSPE), cephalitis and brain infection, glandular fever/chronic lymphocytic leukemia/lymphomas/nasopharyngeal carcinoma, adult T cell leukemia (ATL) and HTLV-I-associated myelopathy/tropical spastic paraparesis (HAM/TSP), a neurologic disease, cytomegalovirus inclusion disease/transplant arterial disease, sexually transmitted infection (STI), oral and cervical cancer/head and neck cancer/squamous cell carcinoma, fever blisters, genital sores and a flu-like illness.

17. The method of claim 16, wherein the disease is hepatitis B.

18. The method of claim 1, wherein the target gene is a gene associated with a disease, whose down regulation ameliorates the disease.

19. The method of claim 18, wherein the target gene is a gene encoding a product selected from the group consisting of VEGF, VEGFR, c-Raf/bcl-2, CEACAM6, EGFR, Bcr-abl, AML1/MTG8, Btk, LPA1, Csk, PKC-theta, Bim1, P53 mutant, stat3, c-myc, SIRT1, ERK1, Cyclooxygenase-2, sphingosine 1-phosphate (SIP) receptor-1, insulin-like growth factor receptor, Bax, CXCR4, FAK, EphA2, Matrix metalloproteinase, BRAF(V599E), Brk, EBV, FASE, C-erbB-2/HER2, HPV E6\E7, Livin/ML-LAP/KIAP, MDR, CDK-2, MDM-2, PKC-α, TGF-β, H-Ras, K-Ras, PLK1, Telomerase, S100A10, NPM-ALK, Nox1, Cyclin E, Gp210, c-Kit, survivin, Philadelphia chromosome, Ribonucleotide reductase, Rho C, ATF2, P110a, P110B of PI 3 kinase, Wt1, Pax2, Wnt4, beta-catanin, integrin, urokinase-type plasminogen activator, Hec1, Cyclophilin A, DNMT, MUC, Acetyl-CoA Carboxylase {alpha}, Mirk/Dyrk1b, MTA1, SMYD3, ACTR, Hath1, Mad2, STK15, XIAP, CD147/EMMPRIN, ENPP2/ATX /ATX-X/FLJ26803/LysoPLD/NPP2/PD-IALPHA/PDNP2, AKT, PrPC, thioredoxin reductase 1, HSPG2, p38 MAP kinase, hTERT, alphaB-Crystallin, STAT6, choline kinase, cyclin D1/CDK4, ASH1, osteopontin, 3-alkyladenine-DNA glycosylase, Plasmalemmal vesicle associated protein-1, SHP2, STAT5, Gab2, Etk/BMX, AFP, Id1/Id3 gene, Maternal embryonic leucine zipper kinase/murine protein serine-threonine kinase 38, phosphatidylethanolamine-binding protein 4, ATP citrate lyase, cyclophilin A, DNA-PK, CT120A, EBNA1, Pim family kinases, hypoxia-inducible factor-1 alpha, acetyl-CoA-carboxylase-alpha, Rac 1/RAC3, Aurora-B, platelet-derived growth factor-D/platelet-derived growth factor receptor beta, Androgen Receptor, EN2, Vav1, BRCA1, Pyk2, leptin, hLRH-1, p28GANK, MCT-1, Fibroblast growth factor receptor 3, p53R2, integrin-linked kinase, cdc42, MAT2A, ICAMs, mimitin, RET, S-phase kinase-interacting protein 2, NRAS, phosphatidylinositol 3-kinase, Fas-ligand, IGFBP-5, E2F4, FLT3, estrogen receptor, LYN kinase, cathepsin B, ZNRD1, ARA55 and activin.

20. The method of claim 19, wherein the target gene is the c-myc gene.

21. The method of claim 1, wherein the target gene is a viral gene.

22. The method of claim 21, wherein the target gene is a gene of a virus selected from the group consisting of human immunodeficiency virus (HIV), hepatitis A virus, hepatitis B virus, hepatitis C virus, hepatitis delta virus, influenza virus, foot-and-mouth disease virus, dengue virus type 2, measles virus, encephalitis virus, Epstein-Barr virus, human T-cell leukemia virus, cytomegalovirus, human papillomavirus and herpes simplex virus.

23. The method of claim 22, wherein the target gene is a gene encoding the polymerase of hepatitis B virus.

24. The method of claim 1, wherein the negative regulators of RNAi are selected from the group consisting of exonucleases and adenosine deaminases.

25. The method of claim 24, wherein the exonuclease is THEX1 or a homolog thereof.

26. The method of claim 24, wherein the adenosine deaminase is ADAR1 or a homolog thereof.

27. The method of claim 1, wherein the target gene is a c-myc gene and the negative regulator of RNAi is THEX1, ADAR1, or a combination thereof.

28. The method of claim 1, wherein the target gene is a gene encoding the polymerase of hepatitis B virus and the negative regulator of RNAi is THEX1.

29. A method of enhancing siRNA efficacy, the method comprising administering to a biological system (i) one or more siRNAs capable of down regulating expression of one or more target genes, and (ii) one or more siRNAs capable of down regulating expression of one or more negative regulators of RNAi.

30. The method of claim 29, wherein the ratio of the siRNAs capable of down regulating expression of target genes to the siRNAs capable of down regulating expression of negative regulators of RNAi is in a range of about 5:1 to about 20:1 (w/w).

31. The method of claim 30, wherein the ratio of the siRNAs capable of down regulating expression of target genes to the siRNAs capable of down regulating expression of negative regulators of RNAi is about 10:1 (w/w).

32. The method of claim 29, wherein the siRNAs capable of down regulating expression of target genes and the siRNAs capable of down regulating expression of negative regulators of RNAi are administered at the same time.

33. The method of claim 29, wherein the siRNAs capable of down regulating expression of target genes are administered after the siRNAs capable of down regulating expression of negative regulators of RNAi have been administered, and still retain their activity.

34. The method of claim 33, wherein the siRNAs capable of down regulating expression of target genes are administered within 3 days after administration of the siRNAs capable of down regulating expression of negative regulators of RNAi.

35. The method of claim 29, wherein the siRNAs capable of down regulating expression of negative regulators of RNAi are administered after the siRNAs capable of down regulating expression of target genes have been administered, and still retain their activity.

36. The method of claim 35, wherein the siRNAs capable of down regulating the expression of negative regulators of RNAi are administered within 3 days after administration of the siRNAs capable of down regulating the expression of target genes.

37. The method of claim 29, wherein all or a portion of the siRNAs are chemically synthesized.

38. The method of claim 29, wherein all or a portion of the siRNAs are synthesized in vivo or in vitro using a nucleic acid sequence.

39. The method of claim 29, wherein all or a portion of the siRNAs are derived in vivo or in vitro from precursor RNAs via chemical modification, biological modification, or combination thereof.

40. The method of claim 29, wherein the target gene is a gene associated with a disease, whose down regulation ameliorates the disease.

41. The method of claim 40, wherein the target gene is a gene encoding a product selected from the group consisting of VEGF, VEGFR, c-Raf/bcl-2, CEACAM6, EGFR, Bcr-abl, AML1/MTG8, Btk, LPA1, Csk, PKC-theta, Bim1, P53 mutant, stat3, c-myc, SIRT1, ERK1, Cyclooxygenase-2, sphingosine 1-phosphate (SIP) receptor-1, insulin-like growth factor receptor, Bax, CXCR4, FAK, EphA2, Matrix metalloproteinase, BRAF(V599E), Brk, EBV, FASE, C-erbB-2/HER2, HPV E6\E7, Livin/ML-IAP/KIAP, MDR, CDK-2, MDM-2, PKC-α, TGF-β, H-Ras, K-Ras, PLK1, Telomerase, S100A10, NPM-ALK, Nox1, Cyclin E, Gp210, c-Kit, survivin, Philadelphia chromosome, Ribonucleotide reductase, Rho C, ATF2, P110a, P110B of PI 3 kinase, Wt1, Pax2, Wnt4, beta-catanin, integrin, urokinase-type plasminogen activator, Hec1, Cyclophilin A, DNMT, MUC1, Acetyl-CoA Carboxylase {alpha}, Mirk/Dyrk1b, MTA1, SMYD3, ACTR, Hath1, Mad2, STK15, XIAP, CD147/EMMPRIN, ENPP2/ATX /ATX-X/FLJ26803/LysoPLD/NPP2/PD-IALPHA/PDNP2, AKT, PrPC, thioredoxin reductase 1, HSPG2, p38 MAP kinase, hTERT, alphaB-Crystallin, STAT6, choline kinase, cyclin D1/CDK4, ASH1, osteopontin, 3-alkyladenine-DNA glycosylase, Plasmalemmal vesicle associated protein-1, SHP2, STAT5, Gab2, Etk/BMX, AFP, Id1/Id3 gene, Maternal embryonic leucine zipper kinase/murine protein serine-threonine kinase 38, phosphatidylethanolamine-binding protein 4, ATP citrate lyase, cyclophilin A, DNA-PK, CT120A, EBNA1, Pim family kinases, hypoxia-inducible factor-1alpha, acetyl-CoA-carboxylase-alpha, Rac1/RAC3, Aurora-B, platelet-derived growth factor-D/platelet-derived growth factor receptor beta, Androgen Receptor, EN2, Vav1, BRCA1, Pyk2, leptin, hLRH-1, p28GANK, MCT-1, Fibroblast growth factor receptor 3, p53R2, integrin-linked kinase, cdc42, MAT2A, ICAMs, mimitin, RET, S-phase kinase-interacting protein 2, NRAS, phosphatidylinositol 3-kinase, Fas-ligand, IGFBP-5, E2F4, FLT3, estrogen receptor, LYN kinase, cathepsin B, ZNRD1, ARA55 and activin.

42. The method of claim 41, wherein the target gene is the c-myc gene.

43. The method of claim 29, wherein the target gene is a viral gene.

44. The method of claim 43, wherein the target gene is a gene of a virus selected from the group consisting of human immunodeficiency virus (HIV), hepatitis A virus, hepatitis B virus, hepatitis C virus, hepatitis delta virus, influenza virus, foot-and-mouth disease virus, dengue virus type 2, measles virus, encephalitis virus, Epstein-Barr virus, human T-cell leukemia virus, cytomegalovirus, human papillomavirus and herpes simplex virus.

45. The method of claim 44, wherein the target gene is a gene encoding the polymerase of hepatitis B virus.

46. The method of claim 29, wherein the negative regulators of RNAi are selected from the group consisting of exonucleases and adenosine deaminases.

47. The method of claim 46, wherein the exonuclease is THEX1 or a homolog thereof.

48. The method of claim 46, wherein the adenosine deaminase is ADAR1 or a homolog thereof.

49. The method of claim 29, wherein the target gene is the c-myc gene and the negative regulator of RNAi is THEX1.

50. The method of claim 29, wherein the target gene is the gene encoding polymerase of hepatitis B virus and the negative regulator of RNAi is THEX1.

51. A composition comprising one or more siRNAs, or precursors thereof, capable of down regulating expression of one or more target genes and comprising one or more siRNAs, or precursors thereof, capable of down regulating expression of one or more negative regulators of RNAi.

52. A composition comprising one or more nucleotide sequences encoding one or more siRNAs, or precursors thereof, capable of down regulating expression of one or more target genes and comprising one or more siRNAs, or precursors thereof, capable of down regulating expression of one or more negative regulators of RNAi.

53. A composition comprising one or more siRNAs, or precursors thereof, capable of down regulating expression of one or more target genes and comprising one or more nucleotide sequences encoding one or more siRNAs, or precursors thereof, capable of down regulating expression of one or more negative regulators of RNAi.

54. A composition comprising one or more nucleotide sequences encoding one or more nucleotide sequences encoding one or more siRNAs, or precursors thereof, capable of down regulating expression of one or more target genes and comprising one or more nucleotide sequences encoding one or more siRNAs, or precursors thereof, capable of down regulating expression of one or more negative regulators of RNAi.

55. The composition of claim 51, wherein the ratio of the siRNAs capable of down regulating expression of target genes to the siRNAs capable of down regulating expression of negative regulators of RNAi is in a range of about 5:1 to about 20:1 (w/w)

56. The composition of claim 51, wherein the ratio of the siRNAs capable of down regulating expression of target genes to the siRNAs capable of down regulating expression of negative regulators of RNAi is about 10:1 (w/w).

57. The composition of claim 51, wherein all or a portion of the siRNAs are chemically synthesized.

58. The composition of claim 51, wherein all or a portion of the siRNAs are synthesized in vivo or in vitro using a nucleic acid sequence.

59. The composition of claim 51, wherein the target gene is a gene associated with a disease, whose down regulation ameliorates the disease.

60. The composition of claim 59, wherein the target gene is a gene encoding a product selected from the group consisting of VEGF, VEGFR, c-Raf/bcl-2, CEACAM6, EGFR, Bcr-abl, AML1/MTG8, Btk, LPA1, Csk, PKC-theta, Bim1, P53 mutant, stat3, c-myc, SIRT1, ERK1, Cyclooxygenase-2, sphingosine 1-phosphate (SIP) receptor-1, insulin-like growth factor receptor, Bax, CXCR4, FAK, EphA2, Matrix metalloproteinase, BRAF(V599E), Brk, EBV, FASE, C-erbB-2/HER2, HPV E6\E7, Livin/ML-IAP/KIAP, MDR, CDK-2, MDM-2, PKC-α, TGF-β, H-Ras, K-Ras, PLK1, Telomerase, S100A10, NPM-ALK, Nox1, Cyclin E, Gp210, c-Kit, survivin, Philadelphia chromosome, Ribonucleotide reductase, Rho C, ATF2, P110a, P110B of PI 3 kinase, Wt1, Pax2, Wnt4, beta-catanin, integrin, urokinase-type plasminogen activator, Hec1, Cyclophilin A, DNMT, MUC1, Acetyl-CoA Carboxylase {alpha}, Mirk/Dyrk1b, MTA1, SMYD3, ACTR, Hath1, Mad2, STK15, XIAP, CD147/EMMPRIN, ENPP2/ATX /ATX-X/FLJ26803/LysoPLD/NPP2/PD-IALPHA/PDNP2, AKT, PrPC, thioredoxin reductase 1, HSPG2, p38 MAP kinase, hTERT, alphaB-Crystallin, STAT6, choline kinase, cyclin D1/CDK4, ASH1, osteopontin, 3-alkyladenine-DNA glycosylase, Plasmalemmal vesicle associated protein-1, SHP2, STAT5, Gab2, Etk/BMX, AFP, Id1/Id3 gene, Maternal embryonic leucine zipper kinase/murine protein serine-threonine kinase 38, phosphatidylethanolamine-binding protein 4, ATP citrate lyase, cyclophilin A, DNA-PK, CT120A, EBNA1, Pim family kinases, hypoxia-inducible factor-1 alpha, acetyl-CoA-carboxylase-alpha, Rac1/RAC3, Aurora-B, platelet-derived growth factor-D/platelet-derived growth factor receptor beta, Androgen Receptor, EN2, Vav1, BRCA1, Pyk2, leptin, hLRH-1, p28GANK, MCT-1, Fibroblast growth factor receptor 3, p53R2, integrin-linked kinase, cdc42, MAT2A, ICAMs, mimitin, RET, S-phase kinase-interacting protein 2, NRAS, phosphatidylinositol 3-kinase, Fas-ligand, IGFBP-5, E2F4, FLT3, estrogen receptor, LYN kinase, cathepsin B, ZNRD1, ARA55 and activin.

61. The composition of claim 60, wherein the target gene is the c-myc gene.

62. The composition of claim 51, wherein the target gene is a viral gene.

63. The composition of claim 62, wherein the target gene is a gene of a virus selected from the group consisting of human immunodeficiency virus (HIV), hepatitis A virus, hepatitis B virus, hepatitis C virus, hepatitis delta virus, influenza virus, foot-and-mouth disease virus, dengue virus type 2, measles virus, encephalitis virus, Epstein-Barr virus, human T-cell leukemia virus, cytomegalovirus, human papillomavirus and herpes simplex virus.

64. The composition of claim 63, wherein the target gene is a gene encoding the polymerase of hepatitis B virus.

65. The composition of claim 51, wherein the negative regulators of RNAi are selected from the group consisting of exonucleases and adenosine deaminases.

66. The composition of claim 65, wherein the exonuclease is THEX1 or a homolog thereof.

67. The composition of claim 65, wherein the adenosine deaminase is ADAR1 or a homolog thereof.

68. The composition of claim 51, wherein the target gene is the c-myc gene and the negative regulator of RNAi is THEX1.

69. The composition of claim 51, wherein the target gene is the gene encoding polymerase of hepatitis B virus and the negative regulator of RNAi is THEX1.

70. A method of determining an optimal ratio of siRNAs capable of down regulating expression of one or more target genes to siRNAs capable of down regulating expression of one or more negative regulators of RNAi in methods for treating a disease or a disorder and in methods for enhancing siRNA efficacy, comprising the following steps:
(a) inducing expression of genes encoding a negative regulator of RNAi using any siRNA molecules;
(b) determining an effective dose of siRNA molecules that is able to induce high expression of the negative regulator of RNAi;
(c) based on the high expression of the negative regulator of RNAi determined in (b), determining a dose of an siRNA that down regulates expression of the negative regulator of RNAi to a base expression level; and
(d) based on the down regulation of the negative regulator of RNAi determined in (c), determining a dose of an siRNA that down regulates expression of one or more target genes.


SEQUENCE LISTING (16 sequences)

SEQ ID NO: 1 - DNA, 28 bp, Artificial Sequence
  Primer
gcgggtaccctgtttgaaggctggattt

SEQ ID NO: 2 - DNA, 30 bp, Artificial Sequence
  primer
atgcgaattctacaggctggaggtggagca

SEQ ID NO: 3 - DNA, 28 bp, Artificial Sequence
  primer
ggaattcgtcttgggtatacatttgacc

SEQ ID NO: 4 - DNA, 25 bp, Artificial Sequence
  primer
ggggtaccagaggacaacagagttg

SEQ ID NO: 5 - DNA, 19 bp, Artificial Sequence
  primer
cggaattcgcagacttgat

SEQ ID NO: 6 - DNA, 20 bp, Artificial Sequence
  primer
ccggtacctggcctcacata

SEQ ID NO: 7 - DNA, 29 bp, Artificial Sequence
  primer
gcgaattcgttccagtactgtgtagcagt

SEQ ID NO: 8 - DNA, 36 bp, Artificial Sequence
  primer
atgcggtaccggatccttgggttcgtgaggaggtcc

SEQ ID NO: 9 - DNA, 28 bp, Artificial Sequence
  primer
gcgaattctctgcactcatcctgagagg

SEQ ID NO: 10 - DNA, 28 bp, Artificial Sequence
  primer
cgggtacctactcctctgcattgtctcc

SEQ ID NO: 11 - DNA, 19 bp, Artificial Sequence
  primer
tgatggactccggtgacgg

SEQ ID NO: 12 - DNA, 20 bp, Artificial Sequence
  primer
tgtcacgcacgatttcccgc

SEQ ID NO: 13 - DNA, 19 bp, Artificial Sequence
  primer
cggaattcgcagacttgat

SEQ ID NO: 14 - DNA, 20 bp, Artificial Sequence
  primer
ccggtacctggcctcacata

SEQ ID NO: 15 - DNA, 29 bp, Artificial Sequence
  primer
gctctagagttccagtactgtgtagcagt

SEQ ID NO: 16 - DNA, 36 bp, Artificial Sequence
  primer
atgcgaattcggatccttgggttcgtgaggaggtcc